(19) United States
(12) Patent Application Publication
(10) Pub. No.: US 2024/0292907 A1
(43) Pub. Date: Sep. 5, 2024

(54) Method for Cleaning an Aerosol Generating Apparatus Using Inkjet Technology

(71) Applicant: JT International SA, Geneva (CH)

(72) Inventors: Alec Wright, Guildford (GB); Eduardo Jose Garcia Garcia, Grand-Saconnex (CH); Grzegorz Aleksander Pilatowicz, Grens (CH)

(73) Assignee: JT International SA, Geneva (CH)

(21) Appl. No.: 18/575,611

(22) PCT Filed: Jun. 28, 2022
(86) PCT No.: PCT/IB2022/055983

§ 371 Date: Dec. 29, 2023

(30) Foreign Application Priority Data
Jun. 30, 2021 (EP) 21182849.6

(51) Int. Cl. A24F 40/85; A24F 40/10; A24F 40/42; A24F 40/50; A24F 40/65; A24F 40/90; B08B 1/14; B08B 9/032

(52) U.S. Cl. CPC A24F 40/85; A24F 40/10; A24F 40/42; A24F 40/50; A24F 40/65; A24F 40/90; B08B 1/14; B08B 9/032; B08B 2209/032

(57) ABSTRACT

A method for cleaning an aerosol generating apparatus of an aerosol smoking system, the aerosol generating apparatus using inkjet technology, the aerosol generating apparatus being configured to generate the aerosol from a smoking liquid provided in a smoking liquid cartridge when the smoking liquid cartridge is connected to the aerosol generating apparatus. The method comprises providing a docking station for the aerosol generating apparatus, providing a cleaning cartridge containing cleaning liquid, whereby the cleaning cartridge is substantially identical to a smoking liquid cartridge in which the smoking liquid is replaced by the cleaning liquid, connecting the cleaning cartridge to the aerosol generating apparatus, inserting the aerosol generating apparatus into a holding mechanism configured to removably hold the aerosol generating device on the docking station: connecting an electrical connector of the docking station to the aerosol generating apparatus such to electrically power a battery charging system of the aerosol generating

### TECHNICAL FIELD

The present invention relates to an inkjet technology aerosol smoking system, more specifically to a method for cleaning an aerosol generating apparatus using this technology.

### BACKGROUND ART

Aerosol smoking systems are configured to generate aerosol as a result of heat generated by electrical power sources, and to deliver aerosol that is intended to be drawn into the mouth of a user. Aerosol smoking systems may provide components of tobacco in an aerosol form. Aerosol smoking systems as used by smokers are commonly referred to as electronic cigarettes. As used herein, the term “aerosol” is meant to include vapors, gases and aerosols of a form or type suitable for human inhalation, whether or not visible, and whether or not of a form that might be considered to be “smoke-like.”

U.S. Pat. No. 10,548,351B2 discloses an example of an aerosol delivery system which makes use of bubble jet technology, more specifically bubble jet heads. A bubble jet head is a component configured to heat a fluid, e.g., an aerosol precursor composition, to produce a bubble of vapor that applies pressure to the fluid and ejects one or more droplets of the fluid therefrom. Bubble jet heads may be defined as including an inlet, e.g., a precursor inlet, an ejection heating element, and a nozzle, e.g., a precursor nozzle, which may be housed within, or defined by, a wafer, substrate, or housing. The aerosol delivery device may include a reservoir at least partially filled with the aerosol precursor composition. Further, the aerosol delivery device may include a bubble jet head in fluid communication with the reservoir. The bubble jet head may be configured to dispense the aerosol precursor composition from the reservoir. Additionally, the aerosol delivery device may include an atomizer configured to heat the aerosol precursor composition dispensed by the bubble jet head to produce an aerosol. The bubble jet head and the atomizer may be fixedly coupled to one another. A typical aerosol delivery system comprises a control body which may be reusable, and a cartridge with the reservoir for the aerosol precursor composition, whereby the cartridge may be disposable. In this regard, whereas certain components within the cartridge may degrade or be depleted, other components such as the bubble jet head within the control body may maintain full functionality despite repeated use.

The aerosol generating system, including the bubble jet head and all related technology is comparatively costly and may under circumstances be used to consume a plurality of capsules. It is therefore reasonable to integrate the bubble jet head in the reusable part of the aerosol smoking system, i.e., the aerosol generation apparatus, rather than including it in a disposable cartridge. This may pose technical design challenges since a die from the bubble jet head or other parts of the bubble jet technology may become soiled with time as the plurality of cartridges are successively consumed. Indeed, for example, smoking liquid can deposit on the die and build up. The smoking liquid attracts other particles and can clog nozzles or create a layer of semi-evaporated fluid with certain components permanently residing on the die(s).

International publication WO 2020/193240A1 relates to a device that allows to consume an aerosol-forming substrate, i.e., a substrate capable of releasing volatile compounds that can form an aerosol, but the publication fails to refer to thermal bubble technology and the use of any smoking liquid capsule. In the publication, the volatile compounds may be released by heating the aerosol-forming substrate. The aerosol formed by heating the aerosol-forming substrate may contain fewer known harmful constituents than would be produced by combustion or pyrolytic degradation of the aerosol-forming substrate. The publication further describes that the device has means for detecting the build-up of aerosol-forming substrate residue on the device. The device accordingly comprises a case controller that receives signals from the residue detection means indicative of the amount of aerosol-forming substrate residue in the cavity or on the heater, and determines an indication of the amount of aerosol-forming substrate residue in the cavity or on the heater based on one or more signal received from the residue detector. A device controller may be configured to receive a cleaning signal from the case controller, in which case the device controller is configured to supply power to the heater in a cleaning cycle. This results in a raising of the temperature of the heater sufficiently to thermally liberate organic material adhered to or deposited in the device cavity or on the heater.

European publication EP3711532A1 relates to a smoking substitute device that includes a cleaning capsule containing a cleaning fluid used to remove the residues from the heating element. In this publication, the cleaning process seems to be very specific in the sense that the heating element is received in the reservoir formed in the cleaning capsule, i.e., it is surrounded by or immersed in the cleaning fluid contained in the reservoir. There seems to be no suggestion that the cleaning capsule is similar to a smoking liquid capsule. In fact, the smokable substance in this publication is not an smoking liquid and could therefore not be provided in a reservoir of a capsule for a liquid. The device is thus in fact not adapted to smoke any smoking liquids, as opposed to a device that uses bubble jet technology.

The invention aims at providing an alternative aerosol smoking system utilizing the inkjet technology, smoking liquid cartridges and a cleaning process, which provides improved hygiene and sustainability of use.

### SUMMARY OF INVENTION

In a first aspect, the invention provides a method for cleaning an aerosol generating apparatus of an aerosol smoking system, the aerosol generating apparatus using inkjet technology, the aerosol generating apparatus being configured to generate the aerosol by heating a smoking liquid provided in a smoking liquid cartridge when the smoking liquid cartridge is connected to the aerosol generating apparatus. The method comprises providing a docking station for the aerosol generating apparatus, providing a cleaning cartridge containing cleaning liquid, whereby the cleaning cartridge is substantially identical to a smoking liquid cartridge in which the smoking liquid is replaced by the cleaning liquid, connecting the cleaning cartridge to the aerosol generating apparatus, inserting the aerosol generating apparatus into a holding mechanism configured to removably hold the aerosol generating device on the docking station; connecting an electrical connector of the docking station to the aerosol generating apparatus such to electrically power a battery charging system of the aerosol generating apparatus, thereby charging the battery; and triggering a triggering mechanism configured to trigger a cleaning process inside the aerosol generating apparatus, whereby the cleaning process involves leading cleaning liquid from the cleaning cartridge to the aerosol generating apparatus of the inkjet technology and evaporate the cleaning liquid.

In a preferred embodiment, the method further comprises exchanging data between the aerosol generating apparatus and a microcontroller of the docking station, the data being at least one of the list comprising: number of puffs since placed in the docking station, fluid changes, battery status.

In a further preferred embodiment, the method further comprises triggering the triggering mechanism by means of the microcontroller in case the microcontroller determines from the data that the cleaning process is due to be executed.

In a further preferred embodiment, the method further comprises activation of the triggering mechanism upon receiving a selection by a user over a selecting device.

In a further preferred embodiment, the method further comprises absorbing any aerosol of cleaning liquid produced during the cleaning process by means of a foam containing cover of the holding mechanism configured to cover a mouthpiece of the aerosol generating apparatus.

In a second aspect, the invention provides a smoking liquid cartridge enabled to contain smoking liquid for an aerosol generating apparatus of an aerosol smoking system using inkjet technology, whereby the liquid is arranged inside the cartridge with a first portion of the liquid being cleaning liquid, which is configured to be fired through a die of the aerosol generating apparatus and to clean it, and a second portion of the liquid being a smoking liquid, which may be evaporated after the cleaning liquid has been used up.

In a third aspect, the invention provides a docking station for cleaning an aerosol generating apparatus of an aerosol smoking system, configured to implement the method described herein above.

By using a cleaning cartridge in the aerosol smoking system utilizing inkjet technology, the invention allows to provide an optimized maintenance, improved hygiene and sustainability of use.

### BRIEF DESCRIPTION OF DRAWINGS

The invention will be better understood through the description of example embodiments, and in reference to the figure, wherein:

Figure shows a printed circuit board of an inkjet technology in conjunction with a smoking liquid cartridge, according to an example embodiment of the invention;

FIG. 2 illustrates an example of a method for cleaning an aerosol generating apparatus according to an example embodiment of the invention; and

FIG. 3 illustrates a flow of air and a flow of smoking liquid through an assembly of smoking liquid cartridge and die, on the way to be nebulized into an aerosol, with a smoking cartridge according to an example embodiment of the invention.

### DESCRIPTION OF EMBODIMENTS

As used herein, the term “smoking liquid” includes materials that provide vaporized components upon heating, typically in the form of an aerosol. “Smoking liquid” includes any tobacco-containing material and may, for example, include one or more of tobacco, tobacco derivatives, expanded tobacco, reconstituted tobacco or tobacco substitutes. “Smoking liquid” also may include other, non-tobacco, products.

“Smoking liquid” may for example be in the form of a liquid. “Smoking liquid” may for example also be a combination or a blend of liquids.

A “smoking liquid cartridge” is an article or component that includes or contains in use the smoking liquid which in use is heated to volatilize.

“inkjet technology” is used herein to designate all components necessary in conjunction with an inkjet head or die required as a part of an aerosol generation apparatus to produce the aerosol from the smoking liquid. The inkjet head or die is configured to heat a fluid, e.g., an aerosol precursor composition, to produce a bubble of vapor that applies pressure to the fluid and ejects one or more droplets of the fluid therefrom. One example of an inkjet technology use is discussed in the above referenced U.S. Pat. No. 10,548,351B2, but this is merely one embodiment of prior art, and many variations and different embodiment may be known from prior art or future developments in the field of inkjet technology for use in aerosol smoking systems. Inkjet technology is understood to encompass various variants such as thermal inkjet, thermal bubble jet technology etc.

There is a need for new easy, more hygienic, and more sustainable aerosol smoking system utilizing the inkjet technology with cartridges of smoking liquid and a process for cleaning thereof.

The present invention provides a method including the use of a docking station in which an aerosol generating apparatus of the aerosol smoking system may be placed and removably held to implement a cleaning of the inkjet technology employed therein to produce the aerosol from a smoking liquid. The smoking liquid is typically provided in a smoking liquid cartridge that may be changed and/or replaced when empty or when a different taste of smoking liquid is desired.

To protect the permanent inkjet technology, and more particularly a die thereof from being blocked due to impurities in the smoking liquid, an aerosol smoking system will typically include a filter placed in a smoking liquid flow circuit just before the die. FIG. 1 illustrates a printed circuit board 100 of an inkjet technology in an aerosol generating apparatus that is configured to carry a pair of dies 101. Hollow needles 103 are configured to conduct smoking fluid (smoking fluid is not illustrated) from a cartridge 104 to the dies 101. An optional filter 102 may be located between the hollow needles 103 and the dies 101 with the aim of filtering the smoking liquid before it reaches the dies 101. It may be expected that with the use of multiple successive cartridges, the filter 102 and/or the dies 101 will accumulate impurities and there is a risk that smoking liquid won't flow at the expected rate anymore. To prevent this, it is possible that each time that the aerosol smoking system is connected to a docking station (docking station not illustrated) where it is electrically charged, it is checked how many cartridges have been used since the last cleaning, and then decided if a filter or die cleaning process should be initiated.

Apart from the filter and the die, it may occur that the aerosol generating apparatus also requires that a cleaning liquid be used to clean and maintain internals of the inkjet technology, e.g., the hollow needles 103, in the best state over time. The storing of the aerosol smoking system in the docking station may be used as an opportunity to run the cleaning liquid through channels of the inkjet technology. In a preferred embodiment, the cleaning liquid may be pure water. The cleaning liquid may also be a base of smoking liquid that doesn't contain any flavoring components nor nicotine, and therefore is particularly pure.

In a preferred embodiment of a cartridge, the cleaning process may be executed with this cartridge as an initial process with a dedicated volume of cleaning liquid before the smoking of the desired smoking liquid starts. This will be detailed below.

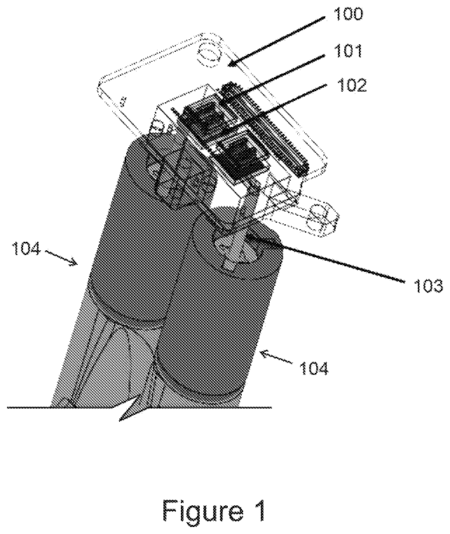

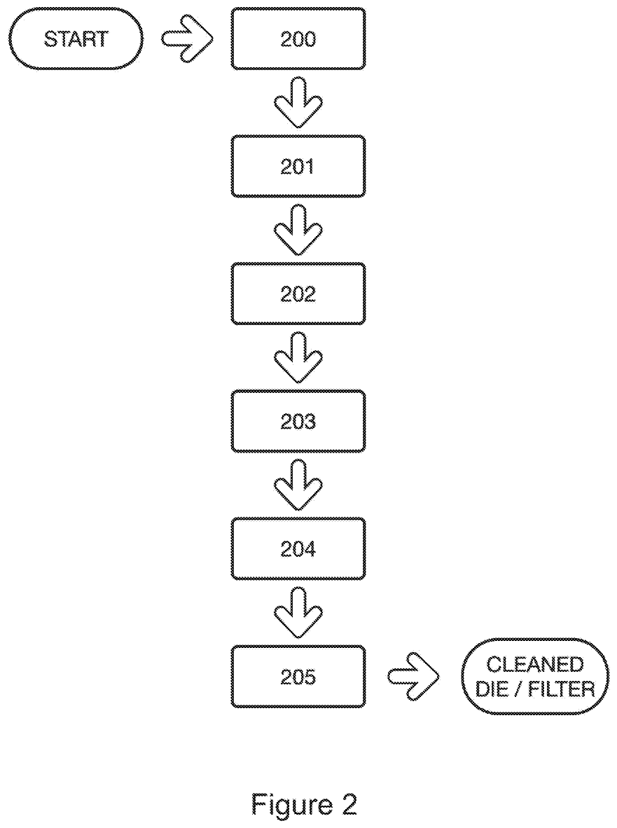

Referring now to FIG. 2, this illustrates an example of the method for cleaning the aerosol generating apparatus of the aerosol smoking system according to an example embodiment of the invention. The aerosol generating apparatus uses inkjet technology. The aerosol generating apparatus is configured to generate the aerosol from a smoking liquid provided in a smoking liquid capsule when the smoking liquid capsule is connected to the aerosol generating apparatus. A step 200 provides a docking station for the aerosol smoking system and its aerosol generating apparatus. A step 201 provides a cleaning cartridge containing cleaning liquid, whereby the cleaning cartridge is substantially identical to a smoking liquid cartridge in which the smoking liquid is replaced by the cleaning liquid. A step 202 connects the cleaning cartridge to the aerosol generating apparatus. A step 203 inserts the aerosol generating apparatus into a holding mechanism configured to removably hold the inhalation device on the docking station. In a step 204 an electrical connector of the docking station is connected to the aerosol generating apparatus such to electrically power a battery charging system of the aerosol generating apparatus, thereby charging the battery. A step 205 involves triggering a triggering mechanism configured to trigger a cleaning process inside the aerosol generating device, whereby the cleaning process involves leading cleaning liquid from the cleaning cartridge to the aerosol generating apparatus of the inkjet technology and evaporate the cleaning liquid.

The docking station may be realized to comprise a housing. In the step 203 of inserting the aerosol generating apparatus into a holding mechanism, both ends and a back of the aerosol generating apparatus are housed by the housing in the docking station, in an upright position. Preferably, a front side of the aerosol generating apparatus remains visible to the user.

In the step 204 of connecting the electrical connector of the docking station to the aerosol generating apparatus, it is possible to make use of well-known standards for the electrical connector, which preferably also integrates data exchange capabilities, such as for example a USB-C interface.

The housing of the docking station may comprise a slidable part, which if configured to slide down over a top end of the aerosol generating apparatus, that typically corresponds to a mouthpiece.

In a preferred embodiment, the method further comprises exchanging data between the aerosol generating device and a microcontroller of the docking station. The exchanged data is at least one of the list comprising: number of puffs since placed in the docking station, fluid changes, battery status. Based on the exchanged data the microcontroller may determine whether the cleaning process is due and execute the step of triggering the triggering mechanism if it is due. The triggering mechanism may however in a further preferred embodiment also be activated upon receiving a selection by a user over a selecting device. The latter may be directly located in the docking station or as part of the aerosol smoking system.

In a further preferred embodiment, the method comprises a step of absorbing any aerosol of cleaning liquid produced during the cleaning process by means of a foam containing cover of the holding mechanism configured to cover a mouthpiece of the aerosol generating apparatus.

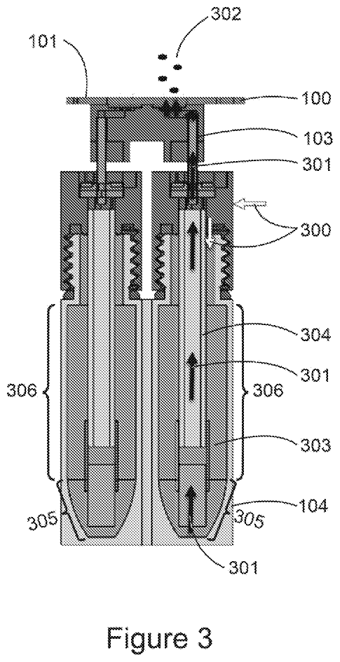

We now refer to FIG. 3 for a further aspect of the invention. FIG. 3 schematically illustrates within the setup of FIG. 1, a flow of air 300 and a flow of smoking liquid 301 through the assembly of smoking liquid cartridge 104 and die 101, with the aim of producing an aerosol 302. The flow of air 300 enters the smoking liquid cartridge 104 and exerts pressure on a surface of the smoking liquid 303 present in the cartridge 104. The smoking liquid 303 is then forced to flow through a central channel 304 of the smoking liquid cartridge 104, and through the hollow needle 103 to reach the die 101 where the smoking liquid is nebulized into the aerosol 302. The smoking liquid cartridge 104 may contain a first portion 305 of the liquid to be cleaning liquid. This is configured to be fired through the die 101 at first and to clean it. The smoking liquid cartridge 104 further contains a second portion 306 of the liquid being smoking liquid, which may only access to the die 101 after the cleaning liquid has been used up, to be evaporated and inhalted.

1. A method for cleaning an aerosol generating apparatus of an aerosol smoking system, the aerosol generating apparatus using inkjet technology, the aerosol generating apparatus being configured to generate the aerosol by heating a smoking liquid provided in a smoking liquid cartridge when the smoking liquid cartridge is connected to the aerosol generating apparatus, the method comprising
providing (200) a docking station for the aerosol generating apparatus,
providing (201) a cleaning cartridge containing cleaning liquid, whereby the cleaning cartridge is substantially identical to a smoking liquid cartridge in which the smoking liquid is replaced by the cleaning liquid,
connecting (202) the cleaning cartridge to the aerosol generating apparatus,
inserting (203) the aerosol generating apparatus into a holding mechanism configured to removably hold the aerosol generating device on the docking station;
connecting (204) an electrical connector of the docking station to the aerosol generating apparatus such to electrically power a battery charging system of the aerosol generating apparatus, thereby charging the battery; and
triggering (205) a triggering mechanism configured to trigger a cleaning process inside the aerosol generating apparatus, whereby the cleaning process involves leading cleaning liquid from the cleaning cartridge to the aerosol generating apparatus of the inkjet technology and evaporate the cleaning liquid.

2. The method of claim 1, further comprising
exchanging data between the aerosol generating apparatus and a microcontroller of the docking station, the data being at least one of the list comprising: number of puffs since placed in the docking station, fluid changes, battery status.

3. The method of claim 2, further comprising
triggering the triggering mechanism by means of the microcontroller in case the microcontroller determines from the data that the cleaning process is due to be executed.

4. The method of any one of claims 1 to 3, further comprising
activation of the triggering mechanism upon receiving a selection by a user over a selecting device.

5. The method of any one of claims 1 to 4, further comprising
absorbing any aerosol of cleaning liquid produced during the cleaning process by means of a foam containing cover of the holding mechanism configured to cover a mouthpiece of the aerosol generating apparatus.

6. A smoking liquid cartridge enabled to contain smoking liquid for an aerosol generating apparatus of an aerosol smoking system using inkjet technology, whereby the liquid is arranged inside the cartridge with a first portion of the liquid being cleaning liquid, which is configured to be fired through a die of the aerosol generating apparatus and to clean it, and a second portion of the liquid being a smoking liquid, which may be evaporated after the cleaning liquid has been used up.

7. A docking station for cleaning an aerosol generating apparatus of an aerosol smoking system, configured to implement the method according to claim 1.
